(19) United States
(12) Patent Application Publication
(10) Pub. No.: US 2012/0122051 A1
(43) Pub. Date: May 17, 2012

(54) Dental Device with Hand-held Instrument and Light Source

(75) Inventors: André Hackel, Biberach (DE); Sven Erdmann, Ulm (DE)

(73) Assignee: Kaltenbach & Voigt GmbH, Biberach (DE)

(21) Appl. No.: 13/291,775

(22) Filed: Nov. 8, 2011

(30) Foreign Application Priority Data
Nov. 11, 2010 (DE) 10 2010 043 795.6

(51) Int. Cl. A61B 6/14

(52) U.S. Cl. 433/29

(57) ABSTRACT

In a dental device with a hand-held instrument at the front end of which there is arranged a removable attachment having at least one light source, the light source is arranged inside a closed housing in the attachment, with current transfer means acting between the attachment and the instrument for supplying power to the light source.

The present invention relates to a dental device with a hand-held instrument which has a light source which is used for observation and/or examination purposes. The present invention relates in particular to a so-called intraoral camera or to a system for the transillumination of teeth.

In medicine, in particular in dentistry, diagnostic systems based on optical principles are increasingly being used. The reason for this is that such devices usually allow a diagnosis to be made without contact, that is to say in particular in a pain-free manner, and, in addition, often also provide optical images with which any necessary therapeutic measures can be communicated to the patient graphically and hence more clearly. For example, so-called intraoral cameras are used in dentistry, which cameras comprise a handpiece, the front end region of which is introduced into the mouth of a patient. In that end region there is generally a light-entry or viewing window for the camera lens, from which the image of the object to be examined is transmitted to an acquisition device, for example a CCD chip.

Such an intraoral camera can further be extended to a system for the transillumination of teeth, as is known inter alia from DE 10 2006 041 020 A1 of the applicant. In that system, the tooth to be examined is irradiated with light within a specific wavelength range, an optical image of the tooth illuminated by the examination radiation then being acquired and evaluated. Because carious areas in the tooth scatter the light differently than healthy dental tissue, such areas can be identified when the tooth is observed with the aid of a camera, it even being possible, if the system is suitably configured, to obtain a more reliable caries diagnosis than is the case with a conventional X-ray examination.

Both in the case of the conventional intraoral camera and in the case of the system for the transillumination of teeth, it is imperative, in order to obtain good and meaningful images, to direct light effectively at the tooth to be observed or examined. At the same time, however, the front end region of the camera or of the hand-held instrument should be of compact form because handling difficulties already arise owing to the confined conditions within the oral cavity of the patient. For that reason it is not usually possible to arrange the light source or sources directly in the front end region of the instrument. In addition to the confined space, the heat generated by the light sources is a further argument against an arrangement in the front end region of the instrument. This cannot be disregarded and could be perceived as unpleasant by the patient, for which reason it was hitherto conventional to arrange the light sources in the more central region of the handle of the instrument and then direct the light to a corresponding light-exit point by means of light guides. This configuration is also advantageous in that the light sources can be arranged inside the instrument protected from external influences. This is important because such examination instruments must regularly be cleaned and sterilized for hygiene reasons, whereby they are in some cases exposed to comparatively high temperatures, which could result in the light sources being damaged.

Because, on the other hand, the transmission of light by means of light guides is always associated with a certain loss of light, it would be desirable to arrange the light sources as close as possible to the light-exit region of the instrument. The object underlying the present invention is to provide a novel solution which permits this and in which at the same time the above-described problems are avoided.

The object is achieved by a dental device having the features of claim 1. Advantageous further developments of the invention are the subject of the dependent claims.

The solution according to the invention is based on the idea of integrating the light source, which can be, for example, an LED or a laser diode, in an attachable attachment of the hand-held dental device, the arrangement of the light source in particular being such that the attachment with the light source as a whole can be sterilized.

To that end, there is proposed according to the present invention a dental device with a hand-held instrument, at the front end of which there is arranged a removable attachment having at least one light source, wherein the light source is arranged inside a closed housing in the attachment and, further, current transfer means acting between the attachment and the instrument are provided for supplying power to the light source.

Because the attachment is removable, it is possible to clean and/or sterilize it separately from the remainder of the dental instrument, so that the particularly sensitive electronic components of the instrument in particular are not exposed to the stresses of the sterilization operation. Furthermore, because the light source is arranged inside a closed housing in the attachment, it is ensured that the light source is likewise not damaged. At the same time, however, it is arranged comparatively close to the light-exit point, so that any losses during transmission by means of light guides are not excessively high. Accordingly, the light delivered by the light source can effectively be used for observation and/or examination purposes.

The light source is preferably arranged on a base which, together with an associated cover, encloses a sealed interior space for accommodating the light source. The cover can have a transparent region for delivery of the light emitted by the light source, the transparent region preferably forming an optical element, in particular a lens for influencing the delivered light.

The current transfer between the instrument and the attachment can be formed, for example, by simple contacts. However, inductive current transfer would also be conceivable, which additionally allows the attachment to be arranged movably, in particular rotatably, relative to the instrument. Because such instruments are generally connected via a supply hose or a cable to a central processing unit or supply unit, handling may in that case be simplified.

The dental device according to the invention can be, for example, a dental camera. However, it can also be used as a device for the transillumination of teeth.

The invention is to be explained in greater detail hereinbelow with reference to the accompanying drawing, in which:

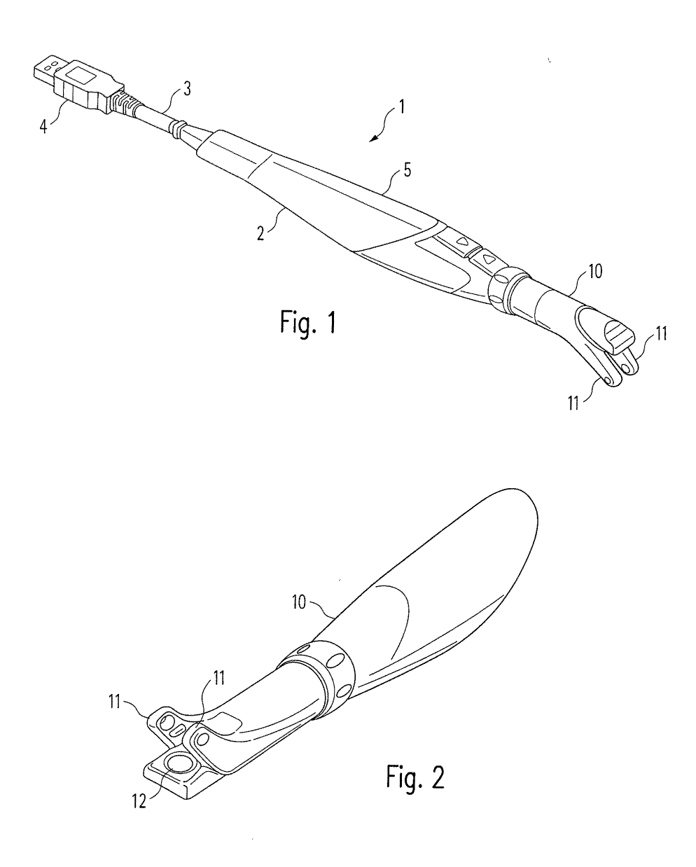

FIG. 1 shows a side view of a dental device according to the invention which is used for the transillumination of teeth;

FIG. 2 shows an enlarged view of the front end region of the device of FIG. 1;

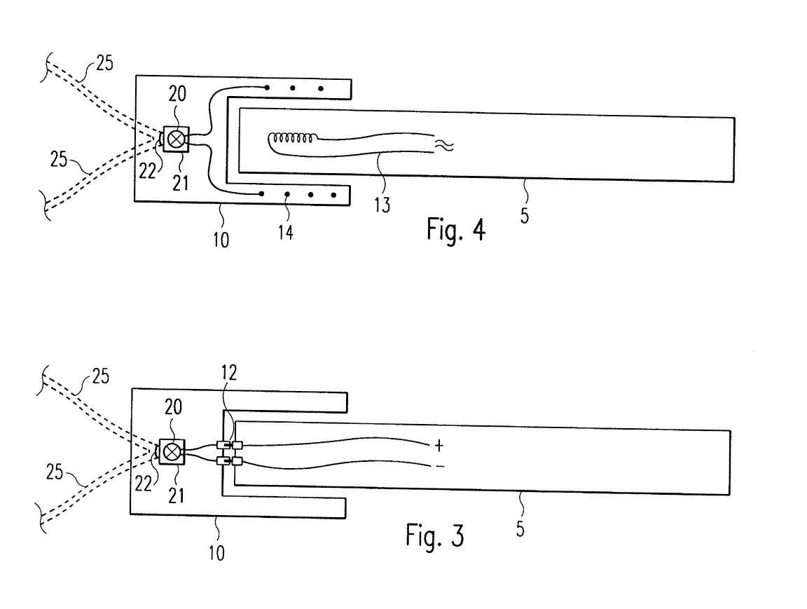

FIG. 3 shows schematically a first variant for current transfer between the instrument and the removable attachment; and

FIG. 4 shows schematically a second variant for current transfer between the instrument and the attachment.

Before the dental device according to the invention is described in more detail, the method on which the examination method is based is first to be explained briefly. The transillumination method is based on passing visible light through a tooth, that is to say transilluminating it. To that end, examination radiation is generated with the aid of a light source and is directed at the tooth. The examination radiation is usually in a wavelength range of approximately from 550 μm to 790 μm, for example approximately 670 μm. The tissue of the tooth does not completely block the examination radiation but instead allows the light to pass through the tooth. The radiation is then partially scattered, carious regions in particular having a characteristic effect on the light. If the tooth transilluminated in that manner is viewed from different viewing directions, such carious regions can be detected because they appear slightly darker. In particular when images taken at different times are compared with one another, caries can thus be detected comparatively effectively and also in good time.

A so-called FOTI (fiber-optic transillumination) device based on this examination principle is shown in FIGS. 1 and 2 and is denoted generally by the reference numeral 1. A fundamental component is a hand-held, elongate instrument 2 which is used to illuminate the tooth to be examined and also serves to acquire the optical image of the illuminated tooth. The data thereby obtained are transmitted via a cable 3, at the end of which there is a USB connector 4, to a central processing unit, in particular a PC (not shown). Here, the images produced are then displayed and the data are evaluated in order to detect caries. Of course, connection with the central unit could also be made by other means, but the USB connector 4 represents a preferred embodiment. The USB connector 4 at the same time also supplies the device 1 with power.

The hand-held instrument 2 consists firstly of an elongate handle 5 in which the fundamental electronic components of the device 1 are arranged. Those components are on the one hand the corresponding electronics for communication with the central unit via the USB connection, as well as means for image acquisition and recording. It can be, for example, a CCD chip with the aid of which an optical image of the tooth is recorded.

Transfer of the optical image to the CCD chip located in the handle 5 is carried out with the aid of an attachment 10, which is removably arranged at the front end of the handle 5. The attachment 10 serves on the one hand to illuminate the tooth 100 to be examined, as is shown in FIG. 2. On the other hand, with the aid of the attachment 10, an image of the illuminated tooth 100 is acquired and passed by optical means to the image acquisition means, that is to say, for example, the CCD chip. To that end, the attachment 10 has at its front end a window directed at the underside, which is coupled via further optical elements to the image acquisition means. For transmission of the image there are used primarily mirrors, prisms, lenses and the like.

Illumination of the tooth 100 is also carried out—as already mentioned—via the attachment 10. To that end, the attachment 10 has at its front end lateral arms 11 which, according to the representation in FIG. 2, are so arranged for the examination of the tooth 100 that they rest on the lateral surfaces of the tooth 100 or the gum, the so-called gingiva. Accordingly, with the aid of the attachment 10 shown, light is coupled laterally into the tooth 100, the tooth then being observed from its upper side. However, it would also be conceivable to configure the attachment in such a manner that light is coupled into the tooth from one side and the tooth is observed from the other side.

For reasons of space, it is not possible to arrange the light source for producing the examination radiation directly in the lateral arms 11. Instead, the light source is located inside the attachment 10 and radiates its light into corresponding light guides, with the aid of which the light is then passed to the arms 11 and there directed at the tooth 100. Because the light source is arranged in the attachment 10, the corresponding light guides can be kept comparatively short, so that the losses resulting therefrom are also kept as small as possible.

Because it is imperative for hygiene reasons that the front region of the device 1, which is introduced into the mouth of the patient, is cleaned and in particular also disinfected regularly, the attachment 10 is removably arranged on the handle 5 of the hand-held instrument 2. However, this in turn means that special measures must be taken in order on the one hand to provide a corresponding power supply for the light source in the attachment 10 and on the other hand to protect the light source adequately in order to prevent damage during the sterilization operation.

To that end, corresponding measures are provided which will be explained in greater detail hereinbelow with reference to FIGS. 3 and 4. Both figures show schematically the arrangement of the attachment 10 on the handle 5.

The solution according to the invention is characterized in that the light source 20, which is preferably formed by a laser diode, is so integrated into the attachment 10 that the attachment can readily be sterilized without any risk of damaging the light source 20. This is achieved by arranging the light source 20 in a hermetically sealed housing 21 which has on its front side a transparent region for delivery of the light emitted by the light source 20. In particular, the transparent region is formed by an optical element in the form of a lens 22. The lens is so arranged in the light path of the light that bundling, or a parallel orientation, of the light beams is achieved, as a result of which they are coupled in an extremely effective manner into the indicated light guides 25, which then branch and lead to the front end regions of the arms 11. The lens 22 can be part of a transparent region of the housing 21 or can form the whole of the transparent region.

Hermetic sealing of the housing 21 is achieved, for example, by arranging the light source 20 on a base which, together with a cover, forms a sealed interior space which is used for accommodating the light source 20.

Power is supplied to the light source 20 via the hand-held instrument 2, there being provided corresponding current transfer means which allow the attachment 10 to be removed. In the variant according to FIG. 3, electrical contacts 12 are used for that purpose, which contacts readily ensure a connection between the handle 5 and the attachment 10. As an alternative, however, an inductive coupling between the device 2 and the handle 5 can also be used—as is shown in FIG. 4. To that end there is located in the handle 5 an exciting coil 13, through which current flows, and in the attachment 10 an induction coil 14, which is electrically connected with the light source 20. This second variant has the advantage that the attachment 10 can be freely rotated relative to the handle 5 of the hand-held instrument 2, which may be advantageous in terms of the handling of the device.

Ultimately, the solution according to the invention achieves the advantage that the light from a light source can be fed very effectively to the required light-exit point, but at the same time it is possible regularly to clean and sterilize the attachment without the risk of damaging the light source.

It is further to be noted that the present invention is not limited to devices or systems for the transillumination of teeth. It would also be conceivable to provide a corresponding configuration in conventional intraoral cameras. In that case too, the above-mentioned advantages arising from the solution according to the invention are achieved.

1. Dental device with a hand-held instrument, at a front end of which there is arranged a removable attachment having at least one light source, wherein the light source is arranged inside a closed housing in the attachment and, current transfer means acting between the attachment and the instrument provided for supplying power to the light source.

2. Dental device according to claim 1, wherein the light source comprises an LED or a laser diode.

3. Dental device according to claim 1, wherein the light source is arranged on a base which, together with a cover, encloses a sealed interior space for accommodating the light source.

4. Dental device according to claim 3, wherein the cover has a transparent region for delivery of light emitted by the light source.

5. Dental device according to claim 4, wherein the transparent region forms an optical element for influencing the delivered light.

6. Dental device according to claim 1, wherein the current transfer means acting between the attachment and the instrument is formed by contacts.

7. Dental device according to claim 1, wherein the current transfer means acting between the attachment and the instrument permit permits inductive current transfer.

8. Dental device according to claim 1, wherein the device is a dental camera.

9. Dental device according to claim 1, wherein the device is a device for the transillumination of teeth.

10. Dental device according to claim 5, wherein the optical element is a lens.
